(19) United States
(12) Patent Application Publication
(10) Pub. No.: US 2014/0276244 A1
(43) Pub. Date: Sep. 18, 2014

(54) Lifestyle Management System

(71) Applicant: MDMBA Consulting, LLC, Encinitas, CA (US)

(72) Inventor: Royan Kamyar, Encinitas, CA (US)

(73) Assignee: MDMBA Consulting, LLC, Encinitas, CA (US)

(21) Appl. No.: 14/207,171

(22) Filed: Mar. 12, 2014

Related U.S. Application Data
(60) Provisional application No. 61/922,949, filed on Jan. 2, 2014.
(60) Provisional application No. 61/780,925, filed on Mar. 13, 2013.

(51) Int. Cl. A61B 5/00; A61B 5/11

(52) U.S. Cl. CPC A61B 5/486; A61B 5/1118; A61B 5/681; A61B 5/1112; A61B 5/7435; A61B 5/7475; A61B 5/0022
USPC 600/595

(57) ABSTRACT

Disclosed herein is a method for a time management system. The method receives inputs from a user where the inputs are for an activity associated with the user. An indication of the activity is displayed on a wearable device at a predetermined time. This displaying is predominate during a normal mode of operation. The method is performed by a software application where a primary focus of the software application is for time management for the user.

### CROSS REFERENCE TO RELATED APPLICATIONS

This application claims priority benefit to U.S. Provisional Application No. 61/780,925, entitled “Lifestyle Management System” filed Mar. 13, 2013, and to U.S. Provisional Application No. 61/922,949, entitled “Lifestyle Management System” filed Jan. 2, 2014, which are incorporated by reference in its entirety herein as if it was put forth in full below.

### BACKGROUND

Recent trends in obesity, diabetes, heart disease, metabolic syndrome, mental health disorders, Alzheimer's disease, and other chronic illnesses have generated increased attention to the importance of lifestyle management for both prevention and treatment of disease. The growing health care economic situation, with spending levels continuing to rise at an unsustainable pace, places additional emphasis on low-cost, high-value lifestyle interventions. The rising percentage of elderly people, combined with the graying of the Baby Boomer generation, has placed an increased attention on wellness and successful aging, which is heavily lifestyle dependent. Meanwhile, there is also a growing general interest from the public toward lifestyle changes intending to make people healthier as well as an ongoing interest from the corporate sector for improving productivity. Many people become distracted from making positive lifestyle changes due to different, unpredictable, and often times stressful inputs from the environment.

For people aspiring to maintain or improve their health and/or productivity levels, it is difficult to design, stay cognizant of, monitor progress and receive ongoing feedback, coaching and support on daily routines. Desired changes to an existing lifestyle are often numerous. Various tools are available in the marketplace to aid a user in managing lifestyle changes.

A user may use paper-based or electronic day planners, calendars and/or to-do lists in order to create a plan, or “formula”, for carrying out all specified lifestyle changes in a given period of time. These are labor intensive and once created, the plan may become virtually “hidden” in whatever planner it was created. For example, a user may have to search a computer or smartphone in order to find their calendar or list to see what activity is planned now or next. Accompanying alarm features, including vibratory, text or email alerts, pop-up windows and/or push notifications on electronic versions interrupt the natural flow of the activities of a user and are ancillary functions of the device being used. These aspects tend to be temporary and hence risk being dismissed or ignored by the user. They may also be perceived as intrusive.

Wearable devices such as a smart-watch to aid a user to manage lifestyle changes are also becoming available in the marketplace. One such electronic device is a wireless watch that may include a variety of services such as a time channel, a messages channel, a contact channel, a calendar channel, a weather channel, a stocks channel, a news channel, a sports channel and/or a games channel.

In the wellness and fitness industry, the market trend is to incorporate multiple functions and broad purpose products. For example, these wearable devices typically track multiple parameters, including number of steps taken, distance traveled, calories burned, sleep patterns and/or heart rate, while also incorporating web portals and software apps with functions ranging from diet and goal logs to games and social networking.

Also available are day-planning, scheduling, calendaring and/or reminder systems with a primarily alphanumeric approach that include pre-planned and post-planned activities in the main display showing ‘past-due’, ‘do it now’, and ‘upcoming’ time segments. These events are to be completed within a pre-determined schedule or routine.

### SUMMARY

Disclosed herein is a method for a time management system. The method receives inputs from a user where the inputs are for an activity associated with the user. An indication of the activity is displayed on a wearable device at a predetermined time. This displaying is predominate during a normal mode of operation. The method is performed by a software application where a primary focus of the software application is for time management for the user.

### BRIEF DESCRIPTION OF DRAWINGS

FIG. 1A is an example embodiment of a flowchart of the method for a time management system

FIG. 1B is an example embodiment of a flowchart of the method for a time management system;

FIG. 1C is an example embodiment of a flowchart of the method for a time management system;

FIG. 2 is an example embodiment for a blank day-planning wheel;

FIG. 3 depicts an example embodiment of the day-planning wheel and activity buttons;

FIG. 4A shows an example embodiment of a wearable device;

FIG. 4B is an example of an alternative embodiment of a wearable device;

FIG. 4C is an example of an alternative embodiment of a wearable device;

FIG. 5 is a sample depiction on the planning interface with editing capabilities;

FIG. 6 is a sample embodiment of the day-planning interface with a merchant mode;

FIG. 7 is an example embodiment for the day-planning wheel with analytics;

FIG. 8 depicts a sample embodiment of the invention on a device;

FIG. 9 depicts a sample embodiment of the invention on a device; and

FIG. 10 illustrates a flowchart of a system for preventing and treating disease while optimizing physiology.

### DETAILED DESCRIPTION

Embodiments of the present invention include a fully integrated lifestyle management method and system for time management with a software-based user interface and a synced hardware. These embodiments aid a user in creating a formula or plan for integrating a given list of desired or recommended activities into a schedule. Once a formula is created, the method or system also aids the user to be cognizant of the decided plan throughout the day.

Disclosed herein is a method for a time management system. The method includes receiving inputs from a user where the inputs are for an activity associated with the user. An indication of the activity is displayed on a wearable device at a predetermined time. This displaying is predominate during a normal mode of operation. The method is performed by a software application where a primary focus of the software application is for time management for the user.

The primary focus of the wearable device may be for time management for the user. The wearable device may be designed to be worn or attached to the user and may be a bracelet, ring, necklace, pendent, wrist watch, smart glasses, small tablet or smartphone.

In one embodiment, the user creates a schedule for the activity. This schedule includes the predetermined time and a second time for a second activity associated with the user. Additional inputs may be provided by a second user. The inputs may be provided by an internet enabled device including a smartphone, a tablet, a laptop or a desktop computer. The inputs may be provided by the wearable device, and the wearable device may be a smartwatch, smartband or smartring. The inputs may be generated by software algorithms based on personal goals, preferences, medical history, biometric or monitoring data. The software application may operate on a web browser, a smartphone application, a tablet application or a smart-watch application.

The indication displayed on the wearable device may be an icon, color or a unique combination of an icon and color. In one embodiment, a second icon may be displayed on the wearable device at a second time where the second icon indicates a second activity associated with the user. In another embodiment, a second color may be displayed on the wearable device at a second time where the second color indicates a second activity associated with the user. In yet another embodiment, a second icon and color combination may be displayed on the wearable device at a second time where the second color and icon combination indicate a second activity associated with the user.

A first icon and a second icon are displayed on the wearable device at the predetermined time. The first icon indicates the activity associated with the user and the second icon indicates a second activity associated with the user. In another embodiment, the activity associated with the user may be a physical activity.

The method further comprises monitoring the user and providing feedback to the user. The feedback indicates compliance with the activity and recommendations or support to achieve desired goals. The monitoring of the user includes biometric sensors, monitors, environmental sensors, or global positioning system devices. The feedback provided to the user includes notifications in real-time or asynchronous time.

A system for time management is also disclosed. The system includes a software application. The software application receives inputs from a user where the inputs are for an activity associated with the user. The system also includes a wearable device. An indication of the activity is displayed on the wearable device at a predetermined time. The displaying is predominate during a normal mode of operation. The primary focus of the software application is for time management for the user.

The primary focus of the wearable device may be for time management for the user. The user creates a schedule for the activity. The schedule includes the predetermined time and a second time for a second activity associated with the user. Additional inputs may be provided by a second user. Inputs may be provided by an internet enabled device including a smartphone, a tablet, a laptop or a desktop computer. The inputs may be provided by the wearable device, and the wearable device may be a smartwatch, smartband or smartring.

FIG. 1A is an example embodiment of a flowchart of the method for a time management system. The method for the time management system 10 begins at step 12 by receiving inputs from a user where the inputs are for an activity associated with the user. At step 14, an indication of the activity is displayed on a wearable device at a predetermined time. This displaying is predominate during a normal mode of operation. The method is performed by a software application where a primary focus of the software application is for time management for the user. FIG. 1B is an example embodiment of a flowchart of the method for a time management system. The time management system 100 can be illustrated by step 110, “Design-A-Day”. Here, the user selects various activities to be performed at predetermined times throughout an entire day. In step 120, this plan is synced to hardware. In step 130, the plan is displayed on a display device. In step 140, the user executes the plan throughout the day. In Step 150, the system (i.e. display device) tracks the progress of the user. In Step 160, the user receives feedback from the system, peers, friends/family, and/or professionals. In step 170, the user repeats this process. FIG. 1C is an example embodiment of a flowchart of the method for a time management system.

In a non-limiting example, a user is diagnosed with pre-diabetes and instructed to undergo radical lifestyle changes in order to avoid life-long medications and the potentially debilitating disease. These recommended changes are numerous and may involve diet, exercise, sleep, stress management, medication and the like. For instance, a user may want to include in a daily routine taking supplements, taking medications, eating five mid-sized, evenly spaced out meals, eating five servings of vegetables and fruits, drinking six cups of water, exercising for 30-60 minutes, meditating for 30-60 minutes, spending time with friends and family and sleeping for seven and a half hours. These changes need to be incorporated into the existing schedule of work, child-care and other obligations.

To create a plan to incorporate all of the desired activities to be performed in one day, a user utilizes the method or system by accessing the design-a-day user interface via software, software application, website or the like. The inputs may be provided by a user using an internet enabled device including a smartphone, a tablet, a laptop or a desktop computer or the like. The inputs may be provided by the wearable device, and the wearable device may be a smartwatch, smartband or smartring. The method or system is performed by a software application and the primary focus of the software application may be for time management for the user. In one embodiment, the software application may operate on a web browser, a smartphone application, a tablet application or a smart-watch application. The user locates a blank 24 hour clock, or a day-planning wheel.

FIG. 2 is an example embodiment for a blank day-planning wheel. The day-planning wheel 210 is shown as empty and an adjacent pane 208 contains a sample set of activity buttons 220 representing a variety of daily activities. These activities may be physical, professional, or personal in nature. In one embodiment, the activity associated with the user may be a physical activity. The user uses the set of activity buttons 220 to sift through a variety of activities and decide which to include in a given 24 hour day. In this way, the inputs are received from a user, the inputs being for an activity associated with a user. The set of activity buttons 220 are shown as circles but may also be depicted as bars, hearts, diamonds and any other shape.

The inputs are received from a device which may be an internet enabled device including a smartphone, a tablet, a laptop or a desktop computer or the like. The inputs may be provided by the wearable device, and the wearable device may be a smartwatch, smartband or smartring. The inner ring 250 of the day-planning wheel 210 shows the time in a 24 hour day and organizes the activities of the user directly corresponding with an outer ring 260. The outer ring 260 of the day-planning wheel 210 contains the activities the user wants to accomplish during the time indicated by the inner ring 250. A sun 270 represents the start of the day for the user while a moon 280 represents the beginning of nighttime. The sun 270 and moon 280 are shown to illustrate a circular clock oriented to the cycle of nature thus providing the user a better sense of time while planning. In another embodiment, the sunrise, moonrise, sunset and moonset are illustrated.

FIG. 3 illustrates an example embodiment for the day-planning wheel. The day-planning wheel 310 has an adjacent pane 308 with a set of activity buttons 320. In one embodiment, the user clicks, drags and drops the selected activity button 320 into the outer ring 360 of the day-planning wheel 310 on the appropriate time of day per the inner ring 350 when the user wishes to perform the activity. Next, the user drags or expands the borders of the time slot to extend the length of time of that specific activity. The length of the circular arc of each activity indicates how long the activity is scheduled for. The lines between the activities represent their duration, as well as when one activity ends and another activity begins. In this way, the user creates a schedule for the activity, which may be a physical activity. The schedule is associated with the user. The user repeats this process until all desired activities to be performed at a particular time throughout the day are included. In one implementation, all the desired activities on the day-planning wheel fit in a 24 hour schedule.

Once a plan is established for a given time period, the user may sync the routine to a display device, such as a wearable device. The wearable device may be designed to be worn or attached to the user and may be a bracelet, ring, necklace, wrist watch, belt buckle, wristband, smart-watch, smart glasses (i.e. Google glass), footwear, pendant, clip-on device or other forms of clothing or jewelry, smartphone and smartphone case. In another embodiment, the device may not be wearable such as a computer, laptop, tablet computer, desk clock, wall clock, decorative orb, other wall or home décor, television, vehicle dashboard, or any other object that is readily visible, audible or tactile by the user. The primary focus of the wearable device may be for time management for the user. In one embodiment, the design-a-day activity may be conducted on the display device itself, i.e. via smartphone. In another embodiment, the user may utilize multiple devices that all sync to the same user account or schedule. In this way, a user may provide inputs by using a combination of a smartphone, a tablet, a laptop or a desktop computer. For example, the user may input a plan while at home from a laptop then update the plan later in the day via smartphone. Or, a single user account may be synched with multiple wearable devices or non-wearable devices such as a watch, ring bracelet, smartphone and wall clock. The tracking data compiled by all of the devices would be synced back to the same single account. Additionally, the user may utilize a single device worn in a variety of ways such as a pendant that may be attached to a necklace, bracelet or worn as a wristband.

In an example embodiment, the wearable device may be designed to be worn on a wrist of the user. FIG. 4A shows an example embodiment of a wearable device, specifically, a smart wristband 400. FIG. 4B is an example of an alternative embodiment of a wearable device, depicting a smart-watch 402. During a normal mode of operation, an indication of the activity at a predetermined time is displayed on a wearable device. The indication may be an icon. The displaying is predominate on the wearable device 400, and 402. The wearable device 400 and 402 acts as a display for the pre-set routine changing at set times throughout the day to correspond to the plan constructed on the day-planning wheel 210. For example, an icon 420 for the main activity is displayed on the wearable device 400 and 402 at the predetermined time where the icon 420 indicates the activity. Also, the icon 420 is predominately displayed on the wearable device 400 and 402 at the predetermined time where the icon 420 indicates the activity.

In another embodiment, a second icon is displayed on the wearable device at a second time where the second icon indicates a second activity associated with the user. Furthermore, a second icon is predominately displayed on the wearable device at a second time where the second icon indicates a second activity associated with the user. This aids the user to be cognizant of the routine throughout the day, and may alert the user to follow the pre-determined plan. In further embodiments, a second color may be displayed on the wearable device at a second time where the second color indicates a second activity associated with the user. Or, a second icon and color combination may be displayed on the wearable device at a second time where the second color and icon combination indicate a second activity associated with the user.

A first icon and a second icon are displayed on the wearable device at the predetermined time. The first icon indicates the activity associated with the user and the second icon indicates a second activity associated with the user. Also, more than two icons may be displayed on the wearable device, or predominately displayed on the wearable device, at a particular time period for varying lengths of time where the icons indicate a third, fourth, or fifth activity associated with the user. This is also illustrated in FIG. 4A.

FIG. 4A shows an example embodiment of a sample depiction on a wearable device 400. The main activity is indicated by the icon 420 which in this case is a sun icon. This is accompanied by additional activities of eating two servings of vegetables and drinking a cup of water. These are indicated by a second icon 430 and a third icon 440, which both depict a carrot icon. A fourth icon 450 is indicated as the drip icon.

FIG. 4B shows an example embodiment of a sample depiction on a wearable device, smart-watch 402. The main activity is indicated by the icon 420 which in this case is a yin-yang icon. This is accompanied by a progress bar 460 showing time elapsed for that activity. FIG. 4C is an example of an alternative embodiment of a wearable device, smart-ring 404. The indication displayed on the wearable device may be an icon, color or a unique combination of an icon and color. The main activity is indicated by the icon 420 which in this case is a sun and a person lying in bed. The smart ring 404 also demonstrates a unique color combination for the current activity.

Referring to FIG. 3, the sleep activity button 370, which is an icon of a person lying in bed, is conveyed to the day-planning wheel by the user. This activity begins at approximately 9:30 pm and ends, hence waking, at 5:00 am for a total of seven and a half hours sleeping. Eventually, the user collects all “to-do” activities on the face of the day-planning wheel 310, and a clear plan or formula is created for executing all desired activities at a predetermined time in a 24 hour day.

For example, according to the day-planning wheel 310 in FIG. 3, the user planned to be asleep at 4:59 am. Hence, the sleep icon is predominately displayed on the wearable device at this time. The user planned to wake according to the day-planning wheel at 5:00 am. Thus, at 5:00 am, the awakening icon is predominately displayed on the wearable device at this time. In one embodiment, the icon and a color combination displayed on the wearable device matches the corresponding activity button entered into the day-planning wheel for that particular time of day. The pattern of activities engrained in the day-planning wheel is thus reflected by the ongoing changes seen in the display device, allowing for easy on-the-go reference by the user. Optionally, a user may create an activity by combining an icon and color using a “Create-a-Button”.

The activity buttons with pictorials such as icons and/or color make the process easy to use for children and the elderly alike and bridges any language barrier. The features of the software application such as limiting added activities to a set time period such as a 24 hour day, allowing for dragging and dropping of different activities and accommodating rapid changes to the schedule to test and view a variety of ideas, makes creating an appropriate 24 hour formula efficient and more enjoyable than traditional methods.

When designing a day using the day-planning wheel, the user may include more than one activity or task for any specified span of time. For example, in one embodiment, the user may have two icons or symbols planned for the time period between 10:00 am and 11:00 am. One icon may be for work while the other icon may be for eating a serving of vegetables. In another embodiment, the user may include three activities or tasks for any specified span of time. For example, one icon may be for drinking a cup of water, a second icon for taking medication and a third icon for taking supplements. FIG. 5 is a sample depiction on the planning interface with editing capabilities. On the day-planning wheel 510, an icon 520 for the main activity of work is indicated by the briefcase icon scheduled from 8:00 am to 4:30 pm. The user schedules a second activity (9:30 am to 11:00 am), a meeting during work, as indicated by a second icon 530, depicted as a pencil icon. Two relaxation breaks are also scheduled during work (8:00 am to 9:30 am), as indicated by the two yin-yang symbols which are third icon 540 and fourth icon 550 respectively.

In one embodiment, the method or system may contain “to-do” list type functionality. For instance, when an activity is performed, the user may touch the screen, swipe a section of the screen, or press a button to indicate that the activity has been completed and is ready to move on to the next activity. In another embodiment, the method or system may display multiple activities at once on the wearable device such as a three by three square of nine total activities and have it function as a “to-do” list. The user may touch the screen, swipe a section of the screen, or press a button each time a task is completed and the corresponding activity may then disappear and/or be replaced by another.

The display of an activity icon on the wearable device may flash at gradual intervals during specified time frames or gestures in order to preserve battery life. In between flashes, the display may be of a traditional time clock, blank or other low-power options. The display may become constant when the wearable device is actively engaged by user, for instance, when the user touches the screen, pushes a button or holds it at a certain position. Users can also “pause” or “skip” or simply turn off the wearable device. In one embodiment, the system may be simplified to only depend on color to demarcate specific activities and represent those to a user on an ongoing basis via the wearable device. For example, the system may simply incorporate seven main colors of the rainbow, of red, orange, yellow, green, blue, indigo and violet. Each color may represent a distinct activity. In the case of practicing lifestyle medicine, the core components of preventive health are exercise, sleep, nutrition, stress management and love/social connection. Each component may be represented by a specific color.

Furthermore, when relayed to the wearable device, the motion, behavior or pattern of the color may provide another layer of semantic meaning or function to the activity being described. For instance, stress management may have a soothing blue tone that gently ebbs and flows when lit, softly coming in and out of focus to the user. Meanwhile, exercise may have a red strong pulsing light that may motivate, energize and alert the user. Thus, along with color, the timing, duration, pattern, repetition and/or period between indications may provide additional context and/or meaning to the visual cue.

Activities planned on the day-planning wheel may not necessarily need to occur daily in the sense that they are to be completed once or more a day. Activities may be sporadic in nature for instance, occurring once every few days at random intervals, or occurring once weekly, once monthly, once yearly, or the like.

When constructing activities on the day-planning wheel, the user may plan or select “modules”. Modules may be compilations of more than one activity under a specified umbrella. For example, a “heart disease” module can incorporate activities for medication, exercise, diet and stress management. If a user designs or selects this, all activities will automatically be incorporated into the day, perhaps at pre-set intervals that may then be adjusted or customized by the user. A user may enter personal goals, guidelines or other parameters that would help adjust the module algorithm to his or her personal preferences, experience, medical history and/or schedule. Modules may also revolve around other categories, such as hobbies. For example, a “surfing” module may include actual time spent surfing as one activity, waxing a board as a separate activity, and monitoring websites for surf conditions as a third activity. Modules may also represent the type of day, for example work day, weekend, or holiday.

Activities may be linked to certain conditions or algorithms chosen and/or set up by the user, third parties and/or the method or system. For example, a “surfing” activity may be dependent on lack of rain for 72 hours and fair to good waves as determined by a surf watch service. If all conditions are met, “surfing” will show up on the day-planning wheel. If conditions are not met, that space on the day-planning wheel may be left blank or replaced by another activity, which may be pre-determined by the user, third party and/or the method.

The user may incorporate flexibility and design open time slots for spontaneity within the day-planning wheel. Examples may include an activity button with a “?”, “mystery”, “TBD” or simply “blank” that gives the user free time to use at will at the given time period.

The user may create customized activity buttons or a set of activity buttons by designing a unique icon and associating it with an activity. This may represent a new activity that is not available. For example, this may encompass a theme. Users may then buy and sell these newly created activity buttons on the website, thus creating a marketplace for new activity buttons. Additionally, the user may program certain messages, songs, videos, or other similar components into the schedule. This may be done to help inspire, motivate and/or educate the user. For example, at the beginning of exercise, perhaps a high energy song will be programmed into the day-planning wheel, and then played on the display device when the corresponding time occurs. Another example is a series of videos on items to buy at the grocery store, ready to play on the display device each time a grocery shopping activity is planned.

The method or system may allow for optimization of the planning of each activity, by adding additional layers of information embedded within each activity. For example, if a meeting activity is planned, there may be additional layers of notes and relevant information embedded within that activity. A user may include notes on location of the meeting with a map, persons involved in the meeting including individualized profile information and photographs, relevant URLs for the meeting, or attach relevant files for the meeting. There may be audio, visual, video or other multimedia reminders or information relevant to the meeting. Such layers of notes and information may be accessed, for example, by a tap on the display of the wearable device, depressing a button on the wearable device, or rotating a dial on the wearable device. If a meal activity is planned, recipes may be listed with serving amounts and nutritional facts, as well as pictures or videos showing sample preparations.

FIG. 5 also shows an example embodiment for editing capabilities. In this embodiment, the adjacent pane 508 is an editing mode allowing capabilities for each activity, such as adjusting start and end time, repeating activities, and adding notes. In another embodiment, the adjacent pane 508 is a reminder mode which includes activities that may span a very brief amount of time, such as less than five minutes, and may be performed either at a specific time or within a specified range of time. In yet another embodiment, the adjacent pane 508 is a secondary activities mode which allows for multi-tasking such as performing the activities in parallel or performing the activities simultaneous, or a higher level of detail regarding the type of activity being performed under the context of another primary activity. In a further embodiment, the adjacent pane 508 is a routine mode allowing users to save and store commonly used routines for future use. For example, the user may have a one day-plan for Mondays, and the user may save and repeat that routine in the future.

Third party platforms, devices or services may be integrated to optimize planning for each activity. For example, data from a third party fitness monitor may be integrated and utilized within an exercise activity. A third party nutrition app may integrate and populate data within a nutrition activity.

Design function may have real-time or asynchronous input from health care professionals, life coaches, therapists, nutritionists, rehabilitation specialists, personal trainers, or any other expert or set of experts. For example, deciding how to plan for daily meals on the day-planning wheel, a user may consult with a dietician telephonically, or via video call, instant messenger, text, email, live chat, or any other communication modality.

Design function may also have automated input from the method or system. The inputs may be generated by software algorithms based on personal goals, preferences, medical history, biometric or monitoring data. A set of algorithms may be provide to produce automated tips and suggestions to the user based on scientific research and/or input from health care professionals, life coaches, therapists, nutritionists, rehab specialists, personal trainers, or any other expert or set of experts. Automated tips and suggestions by system algorithms may be personalized for the user by taking into account personal data, such as age, sex, ethnicity, marital status, number and age of dependents, diagnoses, genomic data, medical history, medications, location, time of year, work schedule, values, goals, or the like. This data may be uploaded from electronic medical records, health records, continuity of care records, or other databases.

Individual experts or groups of experts such as healthcare organizations may package and license advice, input, and/or unique algorithms to the user thus creating a “lifestyle management” marketplace on the website. For example, if a user wants access to a certain hospital or clinic for advice, coaching and/or algorithms in designing activities in a day, a fee may be required. Likewise, a known physician or life coach may also offer services on the website. Services from third parties may include real-time or asynchronous communication, as well as pre-set algorithms based on the particular individual or group style of practice. This may occur as a one-time fee or a subscription service.

Messages, songs, videos, or other similar components may be programmed or recommended into the schedule by health care professionals, life coaches, therapists, nutritionists, rehabilitation specialists, personal trainers, other experts, mentors, peers, spiritual leaders, family and/or friends. In this way, third parties may inspire, motivate or educate the user. The user or third parties may design a series of inspirational, motivational and/or educational messages, songs videos or other similar components that may be packaged, purchased or sold on the website or software application. Inspirational, motivational and/or educational messages, songs, videos or other similar components may be automatically programmed and/or recommended by the method or system. These may be tailored to each user based on personal data.

FIG. 6 is a sample embodiment of the day-planning interface with a merchant mode. The day-planning wheel 610 is shown and the adjacent pane 608 is a merchant mode. The merchant, for instance, an App store, allows the user to purchase a pre-planned routine and/or set of routines. The merchant concept may also be used to distribute designs or themes for depicting activities such as activity buttons in new or stylish ways. In another mode, the adjacent pane 608 is a coaching mode. This is a social, coaching and feedback platform where experts may provide scheduling advice or peers may show support.

FIG. 7 is an example embodiment of the day-planning interface with an analytics mode. The day-planning wheel 710 is shown and the adjacent pane 708 is an analytics mode. The analytics screen consists of trends, reports, tracking results and other useful information. The projected day plan 711 is juxtaposed next to the actual day pattern 712 as determined by the -tracking capabilities of the system. The result is a readily visible side-by-side comparison of how the user planned to spend his or her day and the actual result of what the user did for that particular day. This provides a meaningful platform for professional coaching, feedback and/or intervention. The analytics side bar 708 allows for breakdown and measurement of individual activities and understanding of progress reached towards pre-determined goals. It allows for additional detailed analysis of planned and actual data, and offers graphs, charts and trends. For example, a user may observe that he or she is actually averaging 7 hours a night of sleep over the previous week rather than the planned 8 hours, in a visual, graphical way.

FIG. 8 depicts a sample embodiment of the invention on a device, for example, on a smartphone, watch or clock face. The display is optimized for a smaller screen. In FIG. 8, the full day is seen in a view mode on the day-planning wheel 810, with current time 816 and activity 820 indicated. The wheel icon 830 on bottom of the screen allows the user to switch to an edit mode. In the edit mode, the start and end time of the current activity is indicated and editable with a finger swipe or tap over the numbers. A progress bar may indicate time elapsed. The wheel icon 830 on the bottom of the screen allows the user to switch other modes as well, such as a view mode. The user may swipe the screen left or right or tap the screen to see the next or preceding activity accordingly. In one embodiment, FIG. 9, the outer ring 920 of the day-planning wheel 910 depicts a full day plan using unique colors to represent various pre-planned activities. The inner circle 930 of the day-planning wheel depicts unique icon and color combinations for the current activity. A bookmark symbol may indicate notes embedded under the icon and may be accessed by a swipe of the screen or a single tap on the screen. The user may swipe the screen up or down, left or right or tap the screen to access other modes, such as the edit mode.

In an embodiment where the device is, for example, on a smartphone, watch or clock face a full week of activities may depicted on the screen. In this case, only color is used to demarcate a specific activity. This could also be extended to include an icon. Here, the user can see the entire schedule for the week on one screen. This side-by-side view of the activities may also be used for analytics and tracking, for example, to compare actual performed activities versus planned activities as routines. In this case, day plans may be laid out as vertical or horizontal strips to save space, or as circles.

The method or system may integrate scientific research into automated suggestions for day-planning. This may be tailored in a disease-specific way. For example, with circadian-rhythm disorders, the system may automatically enter or suggest the user to enter sunrise and sunset activity buttons. This population of users is less cognizant of the natural patterns of the solar system and may be able to adjust their lifestyle when using the method or system accordingly. In one implementation, activities therefore do not have to be limited to user-based activities, but can be more broadly defined to include items such as environmental aspects or events such as holidays, concerts, sporting events or the like. The system may automatically adjust the timing of activities such as according to the sunrise and sunset, to correspond to actual variations in sunrise and sunset, for example, with the change of the seasons, without ongoing input from the user.

The method or system may automatically incorporate and integrate third party calendaring systems, such as Microsoft Outlook Calendar, Google Calendar, iCal Apple calendar system, or the like. Data may be exported or imported into the system.

The display device such as the wearable device, may allow for ongoing real-time modification of the pre-set schedule such as “skip”, “cancel”, “snooze”, “pause” or “add” functions. This may be via buttons, touchscreen, gestures, audio command, or the like. The display devices may be synced with the day-planning wheel via Bluetooth, WiFi, NFC, ANT+, zigbee, or other leading technologies. Furthermore, the display device may be a third party device, for example, an iOS smartphone or iWatch, in which case the appropriate system technology is a software application for the third party device.

The method or system further comprises monitoring the user and providing feedback to the user where the feedback indicates compliance with the activity. In one embodiment, the monitoring of the user includes biometric sensors, environmental sensors, monitors and global positioning devices. The display device may contain one or more of a variety of sensors used for vital signs, other biometrics and/or environmental information. These may include, for example, accelerometers, GPS, gyroscopes, altimeter, ambient light sensor, air quality sensor, optical heart rate monitor, other heart rate monitor, blood pressure monitor, respiratory rate monitor, pH monitor, skin conductance monitor, blood sugar monitor, or the like. Additional technologies may be used to provide further data via sensing or monitoring that may aid in tracking and providing feedback. For example, RFID (Radio-frequency identification) technology may be used to identify medications and aid in medication management. NFC (near-field communication) may enable data exchange among devices such as to identify family and friends, or perform relevant transactions.

Monitoring may be accompanied by data analysis to interpret data. For example, based on accelerometers, GPS, gyroscopes and perhaps additional sensors, display devices may decipher what particular activity a user is performing and for how long. This may be enhanced by a machine-learning system, whereby the user wears and utilizes the display device for an extended period of time, and the machine collects data and begins to learn the patterns in the data and links it to various activities (i.e. activity buttons), perhaps with feedback from the user. For example, if the sensor system detects consistent hand and arm motions at 30 to 60 degree angles, with a specific type of repetitive pattern and associates with GPS data pointing to a restaurant, the system may query the user to confirm an eating activity. The machine will then learn this association, and be able to accurately report each time the user is eating at this location and for approximately how long.

Lifestyle medicine vital signs may be defined as sleep, exercise, nutrition, love/social support and stress management. The user may be monitored with a focus on the lifestyle medicine vital signs. Due to the growing importance of these metrics from a global healthcare perspective in the prevention and treatment of chronic disease, there may be a focused optimization of monitoring technologies to report on these specific activities. A convenient denomination with which to track all these activities, given the method or system, would be time such as in hours and minutes, performed on each activity. These may be averaged over a course of days, weeks, months or years, and trends and/or other useful statistics may be garnered and reported. This type of data may be accessed through the day-planning wheel in the analytics mode.

Similarly, given the importance of these lifestyle medicine vital signs, the scheduling side of the method or system may also be optimized around these core activities of sleep, exercise, nutrition, love/social support and stress management. These activities, and related ones, may be placed higher and more prominently on the activity button menu for example. Feedback or scoring systems may also be employed to reward the user for scheduling and/or successfully completion of these core activities.

Feedback may be provided to the user where the feedback indicates compliance with the activity. The feedback provided to the user includes notifications in real-time or asynchronous time. The end result from above would be an accurate activity timeline of the day of the user. This may be the real or actual day routine, as opposed to the desired or planned day routine from the day-planning wheel. In another embodiment, the feedback may be beyond compliance, such as recommendations, motivation, support, congratulations, rewards or points. Comparing these on a website or software application may yield powerful and helpful insights. For example, the method or system may display a series of 24 hour days as vertical strips, side-by-side. Monitoring results may be displayed via charts including the activities performed, time spent doing each activity, etc. Further analyses may take the form of graphs, trends, or the like.

In another embodiment, the system may integrate monitoring data from other, third party sensors and sensor systems including blood pressure monitors, weight scales, pedometers, blood glucose monitors, smartphones and other wearable devices, or the like. Additional smartphone data such as GPS location or a Facebook profile may be utilized to identify location or family and friends, for example.

The user may have a third party such as an expert, group of experts, mentors, peers, spiritual leaders, family and/or friends provide feedback, coaching and/or support for progress towards lifestyle change and management. This may be facilitated by sharing of monitoring data. The third party may observe progress continually over time, and make recommendations for new patterns or activities to increase compliance or reach goals. Support may take the form of text-based support such as text messages, emails, images, video, or audio.

The method or system may provide automated feedback to aid a user to optimize wellness and/or productivity goals. For example, based on the history and biometric data of the user, the method or system may recommend changes to current or future activity. For example, if a user is sensed to have low blood sugar, the display device may automatically change to a snack activity icon. In another example, sensors could monitor the circadian rhythm of the user and provide feedback on optimal activity patterns based on the data of the user and latest science/research. The platform could thus be used to optimize the circadian rhythm of the user for improved or peak health, productivity and performance.

Health care providers and others may assign, prescribe or recommend unique algorithms or routines to users. This may be based on individual data tailored to the patient to prevent or treat certain diseases. Systems may be used to continually prescribe recommended activities or routines based on real-time vital signs, other biometric data and/or historical data such as personal history, goals, preferences, diagnoses or genomic data.

In one implementation, data may be shared with clinicians and others via integration with electronic medical records, patient health record systems or other similar systems. In another implementation, the method or system may integrate with popular social media sites to facilitate peer, family and friend-based motivation, coaching and support.

Schedules may be shared with friends, family, co-workers, clinicians and others. Users may send or share day-plans to or with others and vice versa. Users may have a log of frequently contacted friends and family, or others, and be able to view their day-plans. Users may be able to see their own day-plan and those of a friend or family member, or other, side by side. For example, concentric circles, overlapping or superimposed circles, or vertical or horizontal strips may be implemented. Grouping and sharing of patterns may be used to coordinate family, team or classroom schedules, for example.

User schedules, based on average actual data or personalized goals, may be developed into profiles, either on a particular site or in conjunction with existing social media outlets. These profiles, containing information on personal day-plans, patterns, goals and circadian rhythms may be used to match users with coaches, clinicians, peers, teams, or others for different purposes. Life coaches may be matched to users based on their specific profiles, goals, and/or existing activity patterns, perhaps due to specialty of training and/or experience for example. A simplified example is a user who identifies sleep as a number one priority and is looking for expertise on how to achieve higher quality sleep. A specific clinician, health care professional or life coach may be identified with that specialty, and matched to the user for ongoing coaching and support. Peer-to-peer applications may also exist, for example a group of users looking to incorporate a similar meal plan may be matched by profile data to create a team and provide tips and social support in reaching the goal. “Night owls” and “morning larks”, terms scientists use to identify two major groups of circadian rhythm types, may be identified and grouped for different purposes, i.e. shared goal support.

Historical data may be used to predict or learn patterns in the behavior of the user and display events or activities accordingly. For example, if a user always flosses after brushing their teeth, the system may learn to display the “floss” activity icon after sensing a “brush” activity. This may be accomplished through accelerometers, gyroscopes and other integrated sensors. Alternatively, when the user conveys the brush activity button into the day-planning wheel, a floss activity icon may appear automatically.

The users may be able to implement if/then functions into day planning. For example, if the user actually sleeps less than 8 hours, a nap activity in the afternoon may be supplemented. The day-planning wheel and display device may be updated automatically after processing sensor input.

Messages, songs, videos and other similar components that are programmed or recommended into the schedule by the system to inspire, motivate and/or educate the user may change or adjust over time depending on monitoring input. For example, if a user is sensed to consistently wake up later than a pre-planned set time, a high energy song may be programmed or recommended by the system at the wake-up time.

In one embodiment, additional inputs may be provided by a second user. The user of the method or system may be more than one person. For example, a parent, guardian, clinician, life coach, teacher, employer or caregiver may be controlling the day-planning wheel of the method or system, while the display device is being worn by one or more children, patient(s), client(s), student(s), employee(s) or elderly person(s).

In one embodiment, advertisements may appear on the display device and be appropriately timed for scheduled activities. For example, if the user is scheduled for lunch time, specific food or restaurant ads may appear on the display, perhaps briefly, peripherally, during certain modes of the device or at a push of a button. These ads may be data-enabled, for example incorporating GPS, pre-populated location information or other location data to offer real-time discounts, offers or other form of advertisements/announcements from local restaurants and grocery stores. RFID or NFC (near-field communication) technologies may be enabled to allow for the actual transactions to take place.

In another embodiment, the system may be used at events, conferences, or conventions with long, complicated, or multiple schedules to help disseminate an agenda for a series of planned activities, shows, presentations, or the like.

The method or system may be used to plan time periods other than 24 hours such as minutes, one hour, six hours, months, or any other period of time. For example, the entire day-planning wheel may represent 90 minutes, and be divided into many components or activities to help organize that specified period of time. This may be useful in a classroom, for example, with a busy agenda, and many students working individually or in groups. This may coordinate schedules and/or activities.

In a further embodiment, the method or system may be used as a timer or alarm. For example, for a student taking a test, the day-planning wheel may be set to 90 minutes representing the duration of an exam. For the first 60 minutes, the display can be set to green, signifying plenty of time remaining. With 30 minutes left, the display is set to yellow, and with 5 minutes remaining, the display is set to red. The method or system may also be used for training programs by athletes, monastic or military personnel that may have a strict, pre-determined routine to follow.

Data collected from the user base may be aggregated to provide unique insight into consumer behavior, medical research and public health trends. For example, queries may be conducted to correlate planned (or actual) sleeping times with probability of developing depression. Analytics could also demonstrate which types of scheduling patterns belong to various consumer or demographic segments, to better understand trends and behavior. This aggregated and anonymized data could thus be packaged and sold to third parties who may benefit from this information.

The method or system has a singular core function wherein the primary focus of the software application is for time management for the user through health-based day planning. The wearable device has intelligence geared to this. In the normal mode of operation, the wearable device serves to show the user a planned activity at that particular point in time. In other modes of operation, programmable features or monitoring capabilities on the device may exist in various embodiments. For example, on the device, scrolling to the next or previous activity for potential editing on the go, or tracking progress of planned activities, are possible. These features are secondary to the simplified focus of the overall method or system on time management for the user through health-based day planning. In this way, the user is free from distraction and temptation to engage in unrelated activities such as reading texts or emails, checking sport scores or reading posts on social networks, providing the user who is focusing on a health schedule, a clean interface to do so.

The simplified display represents a counterintuitive departure from industry trends. The main display in its normal mode of operation includes one image matched with one corresponding color. Typically, most current day-planning, scheduling, calendaring and/or reminder systems (both paper and electronic-based) offer complicated displays. The display is normally text-based and includes information extraneous to the central purpose of guiding the user to the pre-planned activity at that moment in time. Constantly reading text and being exposed to extraneous information may be cumbersome and distracting to the user, and allows for less comprehension at a glance. Additionally, the simplified display may be a benefit to those prone to anxiety, as constant exposure to past-due tasks, upcoming tasks, call notifications and text notifications may induce unwanted stress.

The method of using a unique image and color combination to represent a specified activity is included in cognitive science and psychological research showing that these types of simple visual cues are more easily and rapidly processed by the human mind. Many scientists agree that simple visual cues are better at serving as “instant reminders” and “attention grabbers”, especially in our visually-driven culture. By using colors and images, the display is engaging and aesthetically pleasing as opposed to communicating information by text. By depicting time and activities in the form of basic color and image combinations, the display creates a more simple, rapid and user-friendly experience than traditional alphanumeric approaches. This provides a subtle, attractive and positive ongoing nudge for the user to follow the pre-planned, health-based schedule. Thus, a predominate display focused on a desired activity provides significant benefit to a user.

While other wearable devices take advantage of many sensors, efficient data transfer and information processing technologies by providing an increasing array of functions, the present method or system has the primary focus of the wearable device for time management of the user through health-based day planning. Furthermore, while other scheduling systems rely on text and include both pre-planned and post-planned activities predominately displayed on the device, the present method or system utilizes rapidly cognitively processed color and image combinations and predominately displays only the currently planned activity.

In an information and stimulus overloaded society, the need to focus on one or two core tasks throughout the day without unnecessary distraction is growing increasingly difficult and essential. Recent science has shown that the ability of the human mind to multitask is likely overestimated, and human beings are most productive when concentrating on one particular task at a given moment in time. Hence, having a wearable device with a primary focus of the wearable device for time management for the user catering to the importance of focusing on only one activity at a given moment in time is significant. Additionally, given the prevalence of chronic disease and the growing number of elderly, having a tool that aids the user to focus on performing health-based activities becomes beneficial.

The method or system augments the user experience over existing methods or systems by capitalizing on the visual cognitive nature. By depicting time and plans not as a series of uninviting text, but in the form of engaging colors and images, the system attracts the user and creates a more enjoyable experience. Current smart-watches and wearable devices are moving in the opposite direction, trending to include more and more information primarily in the form of text and numbers, while also including images, allowing multiple sources of alerts and notifications, and displaying all the information on the display screen at the same time. With these devices the display is increasingly complicated with a multitude and generality of unrelated functions and applications. This leads to a less satisfactory experience by the user, resulting in increased anxiety due to information and stimulus overload. The method or system is designed to reduce the stress by weeding out less than essential information, by focusing on and displaying only what is important to the user at a particular moment in time.

Chronic disease levels are at an all-time high, with significant human and financial costs. Current modalities for disease prevention, treatment and wellness are fragmented, inefficient and not sufficiently productive. The integrated approach outlined above provides a new, cost-efficient and highly effective means for accomplishing the goals of disease prevention, treatment and wellness.

Over time, many forms of chronic disease prevention, treatment and wellness have been developed. Medications, supplements, medical devices, educational materials, exercise programs, and nutrition counseling are examples of existing solutions.

Due to current disease epidemics of obesity, diabetes, heart disease and depression, among others, a new category of medicine recently emerged called “Lifestyle Medicine”. In 2004 the American College of Lifestyle Medicine was founded as a sub-branch of the American College of Preventive Medicine, and the field was newly established. Lifestyle medicine puts forth the concept of novel vital signs, replacing the traditional ones of heart rate, blood pressure, respiratory rate and pulse oxygen saturation levels, for example. The new “lifestyle vital signs” being recognized may be defined as sleep, exercise, nutrition, stress management and love/social support. Exact metrics, or units for measuring each domain, is yet to be determined, but it is recognized that these are the core aspects of health that need to be addressed, measured and communicated in order for human health to move in a more positive direction.

Meanwhile, another branch of the life sciences is emerging. Chronobiology was traditionally a lab-based basic science, but over recent years and decades has crossed the threshold into greater realms of the clinical space. Evidence is accumulating that human physiology is optimized to pattern itself on the rise and fall of the sun. Almost every major organ system is directly impacted by the sleep-wake cycle, light exposure patterns, feeding patterns and activity patterns that dictate and regulate the circadian rhythm. Traditionally, clinical knowledge and application of this science was limited to the sleep-wake spectrum, but accumulating evidence is proving significant impact on the cardiovascular system, gastrointestinal system, neurological system, endocrine system and musculoskeletal system, and essentially every major physiological system in the body.

Physical trainers of Olympic and professional athletes have become conscious and savvy of the latest science and the human applications in the training of athletes to reach peek physical performance. Meanwhile, there is a shift in medicine due to grass-roots demand, epidemiologic and health care economic issues, that is placing a greater emphasis on wellness versus disease treatment, or how to make healthy people healthier versus focusing solely on helping sick people feel better. A benefit related to chronobiology is assisting people with reaching peak physical performance, as is happening at the professional athletic level, but also peak physiologic and mental performance and overall well-being. The key to optimizing these categories via the circadian rhythm is the appropriate timing of various activities, such as exercise, nutrition and sleep.

Finally, a third great trend in medicine is mindfulness. Mindfulness emphasizes the importance of focus and attention, which is becoming increasingly relevant in a distracted, ADHD plagued society. In 2013, a medical school at a major university required students to practice and study meditation. Over 45 universities have established integrative medicine departments over the last one to two decades. Integrative medicine largely incorporates the concepts of mindfulness, meditation and yoga. Heart disease is still the number one killer in the United States and stress is the main cause of heart disease. Stress management is now recognized as a necessary preventive treatment modality. Mindfulness-based stress reduction is proving to be a leading method of preventing and treating stress.

A singular method of approaching a patient that integrates these three great and powerful trends in medicine is proposed. The focus is on the core elements of lifestyle medicine as defined as exercise, sleep, nutrition, love/social support and stress management. Each of these activities must be successfully performed throughout the day, or most days, in order to achieve their desired preventive or therapeutic effect. This then becomes a challenge of time management, or day-planning for implementation.

To optimize the time management/day-planning implementation of the lifestyle medicine activities, or vital signs, knowledge around chronobiology may be employed. Using circadian rhythm-derived knowledge, a clinician or care provider may wisely, astutely and optimally plan the day for a patient. For example, based on the science collected, a clinician may guide the patient to perform exercise in the afternoon instead of the morning to mitigate risk of injury and to achieve a higher threshold of training.

Because day-planning requires memory and keeping track of a pre-specified plan, a certain level of attention and focus is required throughout the day to stay loyal to a set day-plan. In today's technology-inundated, fast-paced world, it is a challenge. The third component of the system, or novel medical approach, is mindfulness of the day-plan. Mindfulness teaches the principles of re-centering, focusing on the present and re-orienting to what is important, i.e. the priorities of the moment. This serves to re-engage the patient away from a potential distraction and towards the pre-planned goal at hand. By practicing mindfulness in the context of a circadian rhythm-optimized day-plan that integrates all the essential lifestyle medicine components, a new way of practicing medicine that prevents disease, treats disease and optimizes physiology towards wellness is created.

FIG. 9 illustrates a flowchart of a system for preventing and treating disease while optimizing physiology. This is a new path for health and well-being utilized by a user. The flowchart 1000 starts at step 1010, where lifestyle vital signs (LSVS), being sleep, exercise, nutrition, stress management and love/social support, are prescribed. Next, in step 1020, the LSVS are scheduled according to a particular user based on latest research in chronobiology by creating a plan to optimize wellness, prevent or treat disease. Last, in step 1030, mindfulness is practiced by the user to remember and execute the plan. In one embodiment of this system, reminders of the plan are carried out by visual cues. In another embodiment, musical tones serve as reminders. These visual and/or sound cues may be determined by a digital day-planning system. Also, routines or scheduling tactics may be prescribed for various diseases, to prevent or treat, or for wellness and physiological optimization.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the invention. Thus, it is intended that the present subject matter covers such modifications and variations.

1. A method for a time management system, the method comprising:
receiving inputs from a user, the inputs being for an activity associated with the user; and
displaying on a wearable device an indication of the activity at a predetermined time, the displaying being predominate during a normal mode of operation;
wherein the method is performed by a software application; and
wherein a primary focus of the software application is for time management for the user.

2. The method of claim 1, wherein the activity associated with the user is a physical activity.

3. The method of claim 1, wherein the primary focus of the wearable device is for time management for the user.

4. The method of claim 1, wherein the inputs are provided by an internet enabled device including a smartphone, a tablet, a laptop or a desktop computer.

5. The method of claim 1, wherein the inputs are provided by the wearable device, and the wearable device is a smartwatch, smartband or smartring.

6. The method of claim 1, wherein the inputs are generated by software algorithms based on personal goals, preferences, medical history, biometric or monitoring data.

7. The method of claim 1, wherein the user creates a schedule for the activity, the schedule including the predetermined time and a second time for a second activity associated with the user.

8. The method of claim 1, wherein additional inputs are provided by a second user.

9. The method of claim 1, wherein the wearable device is designed to be worn or attached to the user.

10. The method of claim 1, wherein the wearable device is a bracelet, ring, necklace, pendent, wrist watch, smart glasses, small tablet or smartphone.

11. The method of claim 1, wherein the indication is an icon, color or combination of icon and color.

12. The method of claim 11, wherein a second icon is displayed on the wearable device at a second time, the second icon indicating a second activity associated with the user.

13. The method of claim 11, wherein a second color is displayed on the wearable device at a second time, the second color indicating a second activity associated with the user.

14. The method of claim 11, wherein a second icon and color combination is displayed on the wearable device at a second time, the second color and icon combination indicating a second activity associated with the user.

15. The method of claim 1, wherein a first icon and a second icon are displayed on the wearable device at the predetermined time, the first icon indicating the activity associated with the user and the second icon indicating a second activity associated with the user.

16. The method of claim 1, wherein the software application operates on a web browser, a tablet application, a smartphone application or a smart-watch application.

17. The method of claim 1, further comprising:
monitoring the user; and
providing feedback to the user, the feedback indicating compliance with the activity.

18. The method of claim 17, wherein the monitoring of the user includes biometric sensors, monitors, environmental sensors, or global positioning system devices.

19. The method of claim 17, wherein the feedback provided to the user includes notifications in real-time or asynchronous time.

20. A system for time management, the system comprising:
a software application, the software application receiving inputs from a user, the inputs being for an activity associated with the user; and
a wearable device, the wearable device displaying an indication of the activity at a predetermined time, the displaying being predominate during a normal mode of operation;
wherein a primary focus of the software application is for time management for the user.

21. The system of claim 20, wherein the primary focus of the wearable device is for time management for the user.

22. The system of claim 20, wherein the inputs are provided by an internet enabled device including a smartphone, a tablet, a laptop or a desktop computer.

23. The system of claim 20, wherein the inputs are provided by the wearable device, and the wearable device is a smartwatch, smartband or smartring.

24. The system of claim 20, wherein the inputs are generated by software algorithms based on personal goals and preferences, medical history, biometric or monitoring data.

25. The system of claim 20, wherein the user creates a schedule for the activity, the schedule including the predetermined time and a second time for a second activity associated with the user.

26. The system of claim 20, wherein additional inputs are provided by a second user.
